(19) United States
(12) Patent Application Publication
(10) Pub. No.: US 2017/0072113 A1
(43) Pub. Date: Mar. 16, 2017

(54) BIMODAL MOLECULAR WEIGHT COPOLYMERS OF LACTIDE AND GLYCOLIDE

(71) Applicant: ETHICON, INC., Somerville, NJ (US)

(72) Inventors: Sasa ANDJELIC, Nanuet, NY (US); Benjamin D. FITZ, Brooklyn, NY (US)

(21) Appl. No.: 14/854,386

(22) Filed: Sep. 15, 2015

Related U.S. Application Data
(60) Provisional application No. 62/218,050, filed on Sep. 14, 2015.

(51) Int. Cl. A61L 31/06; A61L 27/18; A61L 27/26; A61L 31/14; B29C 35/16; C08L 67/00; C08J 3/00; B29C 49/00; B29C 45/00; A61L 27/58; A61L 31/04

(52) U.S. Cl. CPC A61L 31/06; A61L 27/58; A61L 27/18; A61L 27/26; A61L 31/148; A61L 31/041; C08L 67/00; C08J 3/005; B29C 49/0005; B29C 45/0001; B29C 35/16; A61L 2430/02; A61L 2430/38; A61L 2430/36; A61L 2400/06; C08J 2367/00; C08J 2467/00; B29K 2067/046

(57) ABSTRACT

A bimodal polymer blend of first and second poly(L-lactide-co-glycolide) copolymers, wherein the molecular weight ratio of the first to the second copolymer is at least about two to one, and the blend has crystallization and hydrolysis rates greater than either of the first or second copolymers alone.

### FIELD

This invention relates to absorbable polymer compositions and, more particularly, to bioabsorbable polymer compositions having a bimodal molecular weight distribution, to medical devices produced therefrom and to methods of making bioabsorbable polymer compositions and medical devices.

### BACKGROUND

In polymeric crystals, polymer chains are arranged in a two-dimensional pattern. Due to statistical and mechanical requirements, a complete polymer chain cannot form a single straight stem, the straight stems being limited to a certain length depending on the crystallization temperature. As a result thereof, the stems fold and reenter into a lattice. This reentry can be adjacent to the previous stem or at a random lattice point. The perfectly ordered portion of a polymer is crystalline and the folded surface is amorphous. As such, some polymers are semi-crystalline. The crystalline portion may occur either in isolation or as an aggregate with other similar crystals leading to the formation of mats or bundles or spherulites.

The first step in the formation of spherulites, wherein a straight stem of a polymer chain called a nucleus forms from a random coil, is called nucleation. The rest of the process that includes lamellae growth and spherulite formation is cumulatively called crystal growth. In general, single crystals take the form of thin lamellae that are relatively large in two dimensions and bounded in the third dimension by the folds. Typically all the lamellae within one spherulite originate from a single point. As the spherulite grows, the lamellae get farther and farther apart. When the distance between two lamellae reaches a critical value, they tend to branch. Since the growth process is isotropic, the spherulites have a circular shape in two dimensions and a spherical shape in three dimensions for solidification in a uniform thermal field.

A certain degree of crystallinity is often desired during injection molding or extrusion operations due to the higher thermal and mechanical stability associated therewith. If the crystallization rate is slow or uneven, the resultant product properties may have a wide variation in morphology, creating a potential for lines of imperfection that may lead to material failure and result in lower production capacity and reduced quality of the final product. The ability of a polymer system to crystallize quickly is particularly important for processing, especially for injection molding. The faster that an article crystallizes in a mold, the shorter the cycle time that is needed for developing a morphology that demonstrates increased dimensional stability and avoids warping. While there is an economic benefit in reduced cycle time, shortened cycle times also reduce the time the polymer supply resides in the machine at elevated temperatures. This reduces the amount of thermal degradation such as molecular weight reduction and discoloration that may occur, further improving molded part quality. Retention of molecular weight may additionally lead to better mechanical properties, and in the case of molded parts intended for surgical implantation, the retention of molecular properties post-implantation. The amount of crystallinity needed in the part prior to ejection from the mold depends on the glass transition temperature of the resin as well as the molecular weight of the resin. The lower the glass transition temperature, the higher the level of crystallinity that is needed to provide dimensional stability in a molded part.

In some cases, it is advantageous to have the molded part crystallize outside the mold, that is, after the part has been ejected from the molding machine. The ability for the part to crystallize at a rapid rate is advantageous from a processing standpoint. Rapid crystallization is very helpful in providing dimensional stability of the part as it is undergoing further processing. Besides the rate or kinetics of crystallization, the ultimate level of crystallinity developed in the part is also of great importance. If the level of crystallinity developed in the part is insufficient, the part may not possess the dimensional stability required.

In order to increase the rate of crystallization of a polymer, one must increase either the steady-state concentration of nuclei in the polymer matrix, or increase the rate of crystal growth. In general, an increase in nucleation density can be readily accomplished by adding nucleating agents that are either physical (inactive) or chemical (active) in nature. An introduction of foreign particles can also serve as a nucleation agent. For example, with regard to the absorbable polymers used by the medical industry, such agents can include starch, sucrose, lactose, fine polymer particles of polyglycolide and copolymers of glycolide and lactide, which may be used during manufacturing of surgical fasteners or during subsequent fiber processing. Other ways to increase the nucleation rate without the addition of foreign-based materials include copolymerization with a stiffer, highly crystallizable component, preserving nucleating seeds of a faster crystallizing component during melt manufacturing steps, stress induced nucleation, the use of magnetic field strength or sonic-based energy, as used by the pharmaceutical industry, and the use of specific ratios of mono- to bi-functional initiators in the ring-opening polymerization of glycolide-containing absorbable copolyesters.

With regard to the absorbable polymers having utility in the area of wound management, improved hydrolysis characteristics are often desired to reduce the incidence of infection and increase patient comfort. Improved hydrolysis characteristics are also desired in the area of drug delivery to enhance drug release.

In order to control or increase the bioabsorption/hydrolysis rate of absorbable polymers, several approaches have been proposed. These include exposure to high-energy radiation, such as gamma rays or electron beam radiation treatment under an oxygen atmosphere, blending or copolymerizing the absorbable slow degrading polymer with a faster absorbing material, use of a pore-forming component, varying the pH value of materials having pH sensitive groups and addition of monomers or oligomers to the polymer matrix.

It has been proposed in U.S. Pat. No. 5,539,076 that bimodal molecular weight distributions may be employed for polyolefins to enhance polymer processing, reduce the tendency of die-lip polymer buildup and smoking in on-line operations. Moreover, the crystallization behavior of various binary compositions has been reported for linear polyethylene blends in Polymer, 1998, 29(6), 1045. This study suggests that the two fractions of a binary linear polyethylene blend crystallize separately and independently at moderate and high temperatures and partially co-crystallize at lower temperatures. Similarly, Cheng and Wunderlich, in J. Polym. Sci. Polym. Phys., 1986, 24, 595 and J. Polym. Sci. Polym. Phys., 1991, 29, 515, reported on their crystallization kinetic studies of fractions of poly(ethylene oxides) between 3,500 and 100,000 Mw and their binary mixtures from the melt. These studies suggested that mixed-crystal formation at low crystallization temperatures occurred, with increasing segregation at higher temperatures, despite the higher deposition probabilities of the low molecular weight component.

Von Recum, H. A, Cleek, R. L., Eskint, S. G., and Mikos, A. G., in Biomaterials 18, 1995, 441-447, suggested that modulating lactic acid release during in vivo degradation of PLLA implants by adjusting the polymer polydispersity was feasible. In their work, polydispersed PLLA membranes comprised of blends of monodispersed PLLA of weight average molecular weight of 82500 and 7600 Daltons were fabricated to investigate the effect of polydispersity on degradation characteristics. The PLLA blends exhibited large spherulites of high molecular weight chains embedded in a low molecular weight matrix. During degradation in a phosphate buffer, the release rate of lactic acid increased as the percentage of the low molecular weight component in the blend was increased. For low molecular weight compositions larger than 50%, voids were created in the degrading blends due to the degradation of low molecular weight chains and the concurrent dissolution of lactic acid, and also the release of undegraded particles of high molecular weight.

U.S. Pat. No. 6,488,938 discloses a scleral plug which releases a drug accurately in a specified amount. The scleral plug is formed from a blend of a high-molecular weight polylactic acid having a molecular weight of 40,000 or higher and a low-molecular weight polylactic acid having a molecular weight of 40,000 or lower, and contains a drug for treating or preventing a vitreoretinal disease. The high-molecular weight polylactic acid and the low-molecular weight polylactic acid are in a blending ratio of preferably 90/10 to 50/50, more preferably 90/10 to 70/30, and most preferably 80/20. The molecular weight of the high-molecular weight polylactic acid is preferably 40,000 to 200,000. The molecular weight of the low-molecular weight polylactic acid is preferably 3,000 to 40,000, and more preferably 5,000 to 20,000. The drug is, for example, an antiulcer agent, an antiviral agent, an anti-inflammatory agent, an antifungal agent or an antimicrobial.

U.S. Published Patent Application No. 2013/0225538, incorporated herein by reference in its entirety, and its related applications disclose bimodal bioabsorbable polymer compositions. The compositions include a first amount of a bioabsorbable polymer polymerized so as to have a first molecular weight distribution; a second amount of said bioabsorbable polymer polymerized so as to have a second molecular weight distribution having a weight average molecular weight between about 10,000 to about 50,000 Daltons, the weight average molecular weight ratio of said first molecular weight distribution to said second molecular weight distribution is at least about two to one; wherein a substantially homogeneous blend of said first and second amounts of said bioabsorbable polymer is formed in a ratio of between about 50/50 to about 95/5 weight/weight percent. Also disclosed are a medical device, a method of making a medical device and a method of melt blowing a semi-crystalline polymer blend.

Despite these advances in the art, there is still a need for improved absorbable polymers having increased crystallization and/or hydrolysis rates. Thus, it would be desirable to provide advanced absorbable polymers having increased crystallization and/or hydrolysis rates and methods for their production.

### SUMMARY

Disclosed herein are compositions and methods of enhancing the crystallization and/or hydrolysis rates for absorbable materials. Also disclosed are methods of preparation of absorbable polymer compositions, the compositions so prepared possessing significantly higher crystallization kinetics and/or hydrolysis rates, and devices produced from such compositions. More specifically disclosed herein are absorbable polymeric blend compositions, processes of making the absorbable polymeric blend compositions and medical devices produced from such absorbable polymeric blend compositions.

In one aspect, provided is a bimodal polymer composition, comprising (a) a first amount of a first poly(L-lactide-co-glycolide) copolymer having a first crystallization rate, a first hydrolysis rate and a first molecular weight distribution; and (b) a second amount of a second poly(L-lactide-co-glycolide) copolymer having a second crystallization rate, a second hydrolysis rate and a second molecular weight distribution and a weight average molecular weight from about 10,000 to about 50,000 Daltons; wherein the weight average molecular weight ratio of said first molecular weight distribution to said second molecular weight distribution is at least about two to one; and wherein a substantially homogeneous blend of said first and second copolymers is formed in a ratio of between about 50/50 to about 95/5 weight/weight percent, said substantially homogeneous blend having a crystallization rate greater than each of said first crystallization rate and said second crystallization rate and a hydrolysis rate greater than each of said first hydrolysis rate and said second hydrolysis rate. Advantageously, the first molecular weight distribution is a weight average molecular weight from about 50,000 to about 2,000,000 Daltons.

In another aspect, the bimodal polymer composition can have a heat of fusion value of about 15 to about 50 J/g after melt-processing or heat treating the composition, as measured by differential scanning calorimetry using the heating rate of 10° C./min. In one form, the first copolymer has no measurable crystallinity during the second heating scan, as measured by differential scanning calorimetry at a heating rate of 5° C./min.

Advantageously, the first and second copolymers comprise from about 80 mol % to about 99 mol % L-lactide and about 1 mol % to about 20 mol % glycolide, such as wherein the first and second copolymers comprise about 85 mol % L-lactide and about 15 mol % glycolide, or wherein the first and second copolymers comprise about 95 mol % L-lactide and about 5 mol % glycolide.

In yet another aspect, provided is a bimodal polymer composition, comprising (a) from about 70 wt % to about 80 wt % of a first poly(L-lactide-co-glycolide) copolymer having a first crystallization rate, a first hydrolysis rate and a weight average molecular weight from about 50,000 to about 2,000,000 Daltons; and (b) from about 20 wt % to about 30 wt % of a second poly(L-lactide-co-glycolide) copolymer having a second crystallization rate, a second hydrolysis rate and a second molecular weight distribution and a weight average molecular weight between about 10,000 to about 50,000 Daltons; wherein the weight average molecular weight ratio of said first copolymer to said second copolymer is at least about two to one; and wherein a substantially homogeneous blend of said first and second copolymers has a crystallization rate greater than each of said first crystallization rate and said second crystallization rate and a hydrolysis rate greater than each of said first hydrolysis rate and said second hydrolysis rate.

In still yet another aspect, provided is a medical device comprising a bimodal polymer composition of (a) a first amount of a first poly(L-lactide-co-glycolide) copolymer having a first crystallization rate, a first hydrolysis rate and a first molecular weight distribution; and (b) a second amount of a second poly(L-lactide-co-glycolide) copolymer having a second crystallization rate, a second hydrolysis rate and a second molecular weight distribution and a weight average molecular weight from about 10,000 to about 50,000 Daltons; wherein the weight average molecular weight ratio of said first molecular weight distribution to said second molecular weight distribution is at least about two to one; and wherein a substantially homogeneous blend of said first and second copolymers is formed in a ratio of between about 50/50 to about 95/5 weight/weight percent, said substantially homogeneous blend having a crystallization rate greater than each of said first crystallization rate and said second crystallization rate and a hydrolysis rate greater than each of said first hydrolysis rate and said second hydrolysis rate. Advantageously, the first molecular weight distribution is a weight average molecular weight from about 50,000 to about 2,000,000 Daltons.

Advantageously, the medical device can be one wherein the first and second copolymers of the bimodal polymer composition comprise about 85 mol % L-lactide and about 15 mol % glycolide, said first amount is from about 70 wt % to about 80 wt % and the second amount is from about 20 wt % to about 30 wt %.

In another form, the medical device can be one wherein the bimodal polymer composition thereof can have a heat of fusion value of about 15 to about 50 J/g after melt-processing or heat treating the device over a temperature range of between about 85° C. to about 150° C., as measured by differential scanning calorimetry using the heating rate of 10° C./min.

In preferred forms, the medical device can be a suture, a clip, a staple, a pin, a screw, a fiber, a fabric, a mesh, a clamp, a plate, a hook, a button, a snap, a prosthetic, a graft, an injectable polymer, a vertebrae disc, an anchoring device, a suture anchor, a septal occlusion device, an injectable defect filler, a preformed defect filler, a bone wax, a cartilage replacement, a spinal fixation device, a drug delivery device, a foam or a film.

In another aspect, provided is a method of making a bimodal, semi-crystalline poly(L-lactide-co-glycolide) copolymer blend, comprising blending between about 50/50 to about 95/5 weight/weight percent of (1) a first amount of a first poly(L-lactide-co-glycolide) copolymer having a first crystallization rate, a first hydrolysis rate and a first molecular weight distribution, with (2) a second amount of a second poly(L-lactide-co-glycolide) copolymer having a second crystallization rate, a second hydrolysis rate and a second molecular weight distribution and a weight average molecular weight from about 10,000 to about 50,000 Daltons, wherein the weight average molecular weight ratio of said first molecular weight distribution to said second molecular weight distribution is at least about two to one, said blend has a crystallization rate greater than each of said first crystallization rate and said second crystallization rate and a hydrolysis rate greater than each of said first hydrolysis rate and said second hydrolysis rate, and melt-processing or heat treating the blended copolymers over a temperature range of between about 85° C. to about 150° C., such as by melt blending, extruding, melt spinning, melt blowing or injection molding the blended first and second copolymers at a temperature above their melting temperatures, followed by cooling and crystallizing the blend. Advantageously, the first molecular weight distribution is a weight average molecular weight from about 50,000 to about 2,000,000 Daltons.

In one form of the method, the resulting semi-crystalline poly(L-lactide-co-glycolide) copolymer blend has a heat of fusion value of about 15 to about 50 J/g after melt-processing or heat treating the composition, as measured by differential scanning calorimetry using the heating rate of 10° C./min.

In another form, the first and second copolymers comprise from about 85 mol % to about 95 mol % L-lactide and from about 5 mol % to about 15 mol % glycolide, said first amount is from about 70 wt % to about 80 wt % and the second amount is from about 20 wt % to about 30 wt %.

In yet another aspect, provided is a method of making a medical device, comprising blending between about 50/50 to about 95/5 weight/weight percent of (1) a first amount of a first poly(L-lactide-co-glycolide) copolymer having a first crystallization rate, a first hydrolysis rate and a first molecular weight distribution, with (2) a second amount of a second poly(L-lactide-co-glycolide) copolymer having a second crystallization rate, a second hydrolysis rate and a second molecular weight distribution and a weight average molecular weight from about 10,000 to about 50,000 Daltons, to form a bimodal, blended copolymer, wherein the weight average molecular weight ratio of said first molecular weight distribution to said second molecular weight distribution is at least about two to one, said blend has a crystallization rate greater than each of said first crystallization rate and said second crystallization rate and a hydrolysis rate greater than each of said first hydrolysis rate and said second hydrolysis rate, and forming the medical device by melt-processing or heat treating the blended copolymer over a temperature range of between about 85° C. to about 150° C., such as by melt blending, extruding, melt spinning, melt blowing or injection molding the blended first and second copolymers at a temperature above their melting temperatures, followed by cooling and crystallizing the blend. Advantageously, the first molecular weight distribution is a weight average molecular weight from about 50,000 to about 2,000,000 Daltons.

In one form, the bimodal, blended copolymer of the medical device has a heat of fusion value of about 15 to about 50 J/g after melt-processing or heat treating the composition, as measured by differential scanning calorimetry using the heating rate of 10° C./min.

Advantageously, according to the method the first and second copolymers comprise from about 85 mol % to about 95 mol % L-lactide and from about 5 mol % to about 15 mol % glycolide, said first amount is from about 70 wt % to about 80 wt % and the second amount is from about 20 wt % to about 30 wt %.

In preferred forms, the medical device is a suture, a clip, a staple, a pin, a screw, a fiber, a fabric, a mesh, a clamp, a plate, a hook, a button, a snap, a prosthetic, a graft, an injectable polymer, a vertebrae disc, an anchoring device, a suture anchor, a septal occlusion device, an injectable defect filler, a preformed defect filler, a bone wax, a cartilage replacement, a spinal fixation device, a drug delivery device, a foam or a film.

In another aspect is presented a semi-crystalline polymer composition, comprising a blend of from about 50 to about 95 wt % of a first poly(L-lactide-co-glycolide) copolymer having a first weight average molecular weight distribution; and from about 50 to about 5 wt % of a second poly(L-lactide-co-glycolide) copolymer having a second weight average molecular weight distribution from about 10,000 to about 50,000 Daltons; wherein the ratio of said first molecular weight distribution to said second molecular weight distribution is at least about two to one, and said blend has a crystallization rate greater than crystallization rates of both said first and second copolymers. Advantageously, the first molecular weight distribution is a weight average molecular weight from about 50,000 to about 2,000,000 Daltons.

### BRIEF DESCRIPTION OF THE DRAWINGS

The invention will now be described in more detail with reference to the forms herein disclosed, given only by way of example, and with reference to the accompanying drawings, in which:

FIG. 1 presents differential scanning calorimetry (DSC) traces of the polymers disclosed in Example 4; and

FIG. 2 presents hydrolytic degradation profiles of the polymers disclosed in Example 8.

### DETAILED DESCRIPTION

High molecular weight poly(L-lactic acid) (PLLA) and its high lactide-containing copolymers are known to crystallize quite slowly, if at all, due to the reduced mobility of the highly entangled macromolecules. The crystallinity of different molecular weight PLLA polymers (18,000, 31,000, 156,000 and 425,000 g/mol) has been studied by calorimetric methods (see: Clinical Materials, 1991, 8(1-2), 111). As demonstrated by that study, during cooling from the melt (rate=−0.5° C./min), only the lower molecular weight polymers were able to develop any measurable crystallinity.

The compositions described herein provide significantly higher crystallization rates over the crystallization rates of the individual components. Additionally, the compositions described herein provide significantly higher rates of hydrolysis over the rates of hydrolysis of the individual components of those compositions. Because the inventive blends crystallize faster than controls, under certain conditions the inventive blends possess higher crystallinity levels which can lead to articles having better mechanical properties, such as being stiffer. It will be shown that even when fully annealed, the crystallinity levels of the inventive blends are higher than controls.

The absorbable polymer compositions comprise physical blends of regular-to-high molecular weight poly(L-lactide-co-glycolide) with a lower molecular weight counterpart of the same polymer as a minor component. The polymer blends form semi-crystalline materials which have enhanced processability during melt-processing, including melt blending, extruding, melt spinning, melt blowing or injection molding the blended first and second copolymers at a temperature above their melting temperatures, followed by cooling and crystallizing the blend, due to synergistically faster crystallization kinetics as compared to the individual blend components alone. Binary blends of copolymers described herein also have synergistically higher hydrolysis rates compared to individual components, and may provide more uniform hydrolysis characteristics throughout the polymer matrix.

The presence of the lower molecular weight polymer does not affect the nucleation density of the original material, but greatly increases the growth rate of polymer spherulites. When compositions are produced from the blend of high and low molecular weight poly(L-lactide-co-glycolide) disclosed herein, the rate of crystallization may be at least about 2 times faster than the rate of crystallization over an absorbable polymer made by a substantially similar polymerization process utilizing individual components. Thus, the compositions disclosed herein provide increased crystallization and/or hydrolysis rates as compared to conventional processing, as taken under the same or similar measurement conditions or techniques.

Increased crystallization, as used herein, relates to the improvement in the crystallization properties of a polymer, yielding a polymer that crystallizes at a faster rate. Crystallizing at a faster rate has advantages when melt processing the polymers disclosed herein. This is especially true when fabricating medical devices using an injection molding or fiber extrusion process. Rapid crystallization is particularly advantageous when injection molding articles from resins with low glass transition temperatures, since dimensional stability is usually achieved by crystallization. In the absence of crystallization, injection molded parts made from polymers possessing low glass transition temperatures also frequently display distortion and deformation upon removal from the mold, as they are not able to withstand the forces exerted during the removal process.

As articles crystallize faster, cycle times may be decreased. Not only are there potential economic advantages resulting from the attendant decreased production costs, but faster cycle times also reduce the time the polymer resides in the machine at elevated temperatures. This reduces the amount of degradation that may occur, further improving part quality. The amount of crystallinity needed in the part prior to ejection from the mold depends on the glass transition temperature of the resin as well as the molecular weight of the resin. The lower the glass transition temperature, the higher the level of crystallinity required. It has been found that it is advantageous to have a crystallinity level of at least 10% for some synthetic absorbable polymers possessing low glass transition temperatures. In the case of fibers of higher molecular orientation, the level of crystallinity required is correspondingly higher; at least about 15% and desirably greater than about 25% may be necessary to provide dimensional stability.

Polymers contemplated for use herein include poly(L-lactide-co-glycolide) containing from about 80 mol % to about 99 mol % L-lactide and about 1 mol % to about 20 mol % glycolide, preferably those containing about 85 mol % L-lactide and about 15 mol % glycolide, or those containing about 95 mol % L-lactide and about 5 mol % glycolide. D,L-lactide cannot be used as the lactide component, since forms amorphous polymers which do not crystallize. Additionally, it is known that other common copolymers of lactide and glycolide, such as 50/50 mol % lactide/glycolide, are likewise amorphous.

In a first disclosed form, the polymer blends described herein include homogenous physical mixtures of the same polymers having two distinct molecular weight distributions, wherein a weight average molecular weight ratio of the first molecular weight distribution to the second molecular weight distribution is at least or greater than about two to one. Preferably, this ratio may be about three to one, more preferably in the range of about four to six to one.

As indicated above, the polymer blends disclosed herein are two component blends of a bioabsorbable poly(L-lactide-co-glycolide), each component selected on the basis of its weight average molecular weight distribution. The first component is selected to possess a weight average molecular weight between about 50,000 to about 2,000,000 Daltons. The second component is selected to possess a weight average molecular weight between about 10,000 to about 50,000 Daltons.

In another form, the composition comprises a two component poly(L-lactide-co-glycolide) blend having a first component of a weight average molecular weight between about 50,000 to about 1,000,000 Daltons, preferably between about 80,000 to about 500,000 Daltons, and a second component of a weight average molecular weight between about 20,000 to about 45,000 Daltons.

The amounts of the first and the second molecular weight distributions is preferably in ratios to each other of between about 50/50 to about 95/5 (weight/weight) percent, respectively. More preferably, this ratio is between 70/30 and 95/5, respectively. Particularly suitable are bimodal compositions having weight ratios of higher to lower molecular weight distributions of 75/25 and 80/20, respectively. For example, the first, higher molecular weight distribution copolymer can comprise from about 70 wt % to about 80 wt %, and the second, lower molecular weight distribution copolymer amount can comprise from about 20 wt % to about 30 wt %, based on the weight of the combined copolymers as a whole.

The composition is capable of crystallizing in the range of between about 110° C. to about 135° C., as verified by calorimetric measurements. Similarly, the rate of hydrolysis of the composition, measured in distilled water at a constant pH value is at least 30% or greater than the rate of hydrolysis exhibited by either the first or second polymer component alone, as evaluated using an absorption profiler instrument.

The bimodal copolymer blends of the invention can display a heat of fusion (which is directly proportional to degree of crystallinity) from about 15 to about 40 J/g after melt-processing or heat treating the composition over a temperature range of between about 85° C. to about 150° C., even when the higher molecular weight copolymer has no measurable crystallinity, as measured by differential scanning calorimetry during the second heat measurements at a constant heating rate of 5° C./min.

In accordance herewith, a medical device may be produced from a blended absorbable polymeric composition disclosed herein exhibits substantially increased rates of hydrolysis and/or crystallization, as compared to the rate of hydrolysis and/or crystallization of a device produced from an individual polymeric component of the blended composition. The medical devices contemplated herein include those selected from the group consisting of sutures, clips, staples, pins, screws, fibers, stents, gel caps, tablets, microspheres, meshes, fabrics, clamps, plates, hooks, buttons, snaps, prosthetics, grafts, injectable polymers, vertebrae discs, anchoring devices, suture anchors, septal occlusion devices, injectable defect fillers, preformed defect fillers, bone waxes, cartilage replacements, spinal fixation devices, drug delivery devices, foams and films.

The blended compositions disclosed herein may further comprise a pharmaceutically active agent substantially homogenously mixed with the copolymer blend of the present invention. It is envisioned that the pharmaceutically active agent may be released in a living body organism by diffusion and/or a polymer hydrolysis mechanism.

The pharmaceutically active agent may be selected from the group consisting of analgesics, anti-inflammatory compounds, muscle relaxants, anti-depressants, anti-viral, antibiotic, anesthetic, and cytostatic compounds. In another form, the analgesics may include acetaminophen or ibuprofen. In yet another form, the anti-inflammatory compounds include compounds selected from the group consisting of non-steroidal anti-inflammatory drugs (NSAIDs), prostaglandins, choline magnesium salicylate, salicyclic acid, corticosteroids, methylprednisone, prednisone, and cortisone.

The method of making the bimodal compositions disclosed herein may, in general, comprise a step of blending a first poly(L-lactide-co-glycolide) component having a first molecular weight distribution with a second poly(L-lactide-co-glycolide) component having a second molecular weight distribution. In one form, the blending step is performed by melting the amounts of first and second components in a sufficient quantity at a temperature above the melting point of the highest melting component, so as to ensure forming a substantially homogenous mixture. In another form, the blending step is performed by dissolving the amounts of first and second molecular weight distributions in a sufficient quantity in a suitable solvent, and subsequently, removing the solvent, thereby forming a substantially homogenous mixture. The dissolving step of the method may further comprise selecting a suitable solvent from the group consisting of acetone, ethyl acetate, ethyl lactide, tetraglycol, chloroform, tetrahydrofuran, dimethyl sulfoxide, N-methylpyrollidinone, dibutyl phthalate, methylene chloride, methyl ethyl ketone, dibasic esters, methyl isobutyl ketone, dipropylene glycol, dichloromethane and hexafluoroisopropyl alcohol.

Specific embodiments of the present invention will now be described further, by way of example. While the following examples demonstrate certain embodiments of the invention, they are not to be interpreted as limiting the scope of the invention, but rather as contributing to a complete description of the invention.

### EXAMPLES

Several commercially available instruments were utilized. A description of the equipment used follows.

### Differential Scanning Calorimetry (DSC)

Overall crystallization rates depend principally on two factors: the concentration of growing spherulites over time (nucleation rate) and the rate of spherulitic growth. As expected, these processes have a measurable effect on calorimetric data. Calorimetric results were generated on a TA Instruments Differential Scanning Calorimeter, Model 2910 MDSC, using dry N₂ as a purge gas.

Crystallization studies were conducted using the second heat measurements in the following manner: Non-isothermal DSC crystallization data were obtained for several poly(L-lactide-co-glycolide) polymers after first, melting the copolymers at about 185° C. to about 200° C., second, quenching the copolymers to about −20° C. or below, and third, conducting the heating step at a constant heating rate of 5-10° C./min. Again, a dramatic increase in the heat of fusion values (i.e. crystallization rates) were observed for the two blends compared to both poly(L-lactide-co-glycolide) homopolymers using this non-isothermal method.

### Hydrolysis Profile

The hydrolysis profile method determines the hydrolytic degradation time of ester-containing samples. The hydrolysis profile is generated by first hydrolytically degrading a test specimen, while maintaining a constant pH by titrating with a standard base and measuring the quantity of base used with time. This measurement and titration procedure is automated through the use of a pH stat instrument (718 STAT Titrator Complete, by MetroOhm, using Software TiNet 2.4). The samples are placed in a 70 mL stirred, sealed, bath of deionized water held at 70° C.+/−0.2° C. and at a pH of 7.3. Each sample bath is continuously monitored for pH changes (drops in pH) from the set point of 7.3. If any decrease is measured, a sodium hydroxide solution is added to return to the bath 7.3 (NaOH 0.05N). The following measurements are recorded by computer: temperature, volume of base added (V(t)), and pH, over time. The V(t) time-course is analyzed to yield the time to 50% hydrolysis, t 50. Prior to each sample run, the pH probe at each test station is calibrated at pH values of 4.0, 7.0 and 10.0, using standard solutions.

### Example 1

### Synthesis of 85/15 Poly(L(−)-lactide-co-glycolide): Standard Molecular Weight Polymer

Into a suitable 15-gallon stainless steel oil jacketed reactor equipped with agitation, 43.778 kg of L(−)-lactide and 6.222 kg of glycolide were added along with 121.07 g of dodecanol and 9.02 mL of a 0.33M solution of stannous octoate in toluene. The reactor was closed and a purging cycle, along with agitation at a rotational speed of 12 RPM in an upward direction, was initiated. The reactor was evacuated to pressures less than 200 mTorr followed by the introduction of nitrogen gas to a pressure slightly in excess of one atmosphere. The cycle was repeated several times to ensure a dry atmosphere.

At the end of the final introduction of nitrogen, the pressure was adjusted to be slightly above one atmosphere. The vessel was heated at a rate of 180° C. per hour until the oil temperature reached approximately 130° C. The vessel was held at 130° C. until the monomer was completely melted and the batch temperature reached 110° C. At this point the agitation rotation was switched to the downward direction. When the batch temperature reached 120° C., the agitator speed was reduced to 7.5 RPM, and the vessel was heated using an oil temperature of approximately 185° C., with a heat up rate of approximately 60° C. per hour, until the molten mass reached 180° C. The oil temperature was maintained at approximately 185° C. for a period of 2.5 hours.

At the end of the reaction period, the agitator speed was reduced to 5 RPM, the oil temperature was increased to 190° C., and the polymer was discharged from the vessel into suitable containers for subsequent annealing. The containers were introduced into a nitrogen annealing oven set at 105° C. for a period of approximately 6 hours; during this step the nitrogen flow into the oven was maintained to reduce degradation due to moisture.

Once the annealing cycle was complete, the polymer containers were removed from the oven and allowed to cool to room temperature. The crystallized polymer was removed from the containers, bagged, and placed into a freezer set at approximately −20° C. for a minimum of 24 hours. The polymer was removed from the freezer and placed into a Cumberland granulator fitted with a sizing screen to produce polymer granules of approximately 3/16 inches in size. The granules were sieved to remove any “fines” and then weighed. The net weight of the ground polymer was 39.46 kg, which was then placed into a 3 cubic foot Patterson-Kelley tumble dryer.

The dryer was closed and the pressure is reduced to less than 200 mTorr. Once the pressure was below 200 mTorr, tumbler rotation was activated at a rotational speed of 8-15 RPM and the batch was vacuum conditioned for a period of 10 hours. After the 10 hour vacuum conditioning, the oil temperature was set to a temperature of 120° C., for a period of 32 hours. At the end of this heating period, the batch was allowed to cool for a period of at least 4 hours, while maintaining rotation and high vacuum. The polymer was discharged from the dryer by pressurizing the vessel with nitrogen, opening the slide-gate, and allowing the polymer granules to descend into waiting vessels for long term storage. The long term storage vessels were air tight and outfitted with valves allowing for evacuation so that the resin was stored under vacuum.

The resin was characterized. It exhibited an inherent viscosity of 1.79 dL/g, as measured in hexafluoroisopropanol at 25° C. at a concentration of 0.10 g/dL. Gel Permeation Chromatography (GPC) revealed a weight average molecular weight of about 90,000 Daltons. Differential Scanning Calorimetry (DSC) using a heating rate of 10° C./min revealed a glass transition temperature of 59° C. and a melting transition of 150° C., with the heat of fusion about 35 J/g. Nuclear magnetic resonance (NMR) analysis confirmed that the resin was a random copolymer of polymerized L(−)-lactide and glycolide (Lac/Gly), with a composition of about 85 percent L(−)-lactide and about 15 percent glycolide on a molar basis.

### Example 2

### Synthesis of 85/15 Poly(L(−)-lactide-co-glycolide): Lower Molecular Weight Polymer

Into a suitable 2-gallon stainless steel oil jacketed reactor equipped with agitation, 5.253 kg of L(−)-lactide and 0.747 kg of glycolide were added along with 48.43 g of dodecanol and 1.08 mL of a 0.33M solution of stannous octoate in toluene. The reactor was closed and a purging cycle, along with agitation at a rotational speed of 25 RPM in an upward direction, was initiated. The reactor was evacuated to pressures less than 200 mTorr followed by the introduction of nitrogen gas to a pressure slightly in excess of one atmosphere. The cycle was repeated several times to ensure a dry atmosphere.

At the end of the final introduction of nitrogen, the pressure was adjusted to be slightly above one atmosphere. The vessel was heated at a rate of 180° C. per hour until the oil temperature reached approximately 130° C. The vessel was held at 130° C. until the monomer was completely melted and the batch temperature reached 110° C. At this point the agitation rotation was switched to the downward direction. When the batch temperature reached 120° C., the agitator speed was reduced to 20 RPM, and the vessel was heated using an oil temperature of approximately 185° C., with a heat up rate of approximately 60° C. per hour, until the molten mass reached 180° C. The oil temperature was maintained at approximately 185° C. for a period of 2.5 hours.

At the end of the reaction period, the agitator speed was reduced to 4 RPM, the oil temperature was increased to 190° C., and the polymer was discharged from the vessel into suitable containers (aluminum pie plates) for subsequent annealing. The annealing, drying, and grinding procedures were conducted using the same approach as described earlier in Example 1.

The resulting dried copolymer 85/15 poly(L(−)-lactide-co-glycolide) resin had a glass transition temperature of 54° C., a melting point of 152° C., and an enthalpy of fusion of 42 J/g, as measured by DSC using a heating rate of 10° C./min. The resin has a weight average molecular weight of 41,000 Daltons as determined by GPC method, and exhibited an inherent viscosity of 0.83 dL/g, as measured in hexafluoroisopropanol at 25° C. at a concentration of 0.10 g/dL. Nuclear magnetic resonance analysis confirmed that the resin is a random copolymer of polymerized L(−)-lactide and glycolide, with a composition of about 85 percent L(−)-lactide and about 15 percent glycolide on a molar basis.

### Example 3

### Dry Blending of Unimodal Lactide/Glycolide Copolymers

Appropriate amounts of the 85/15 lactide/glycolide copolymers of standard (Example 1) and lower weight average molecular weight (Example 2), both in divided form (ground), were combined in dry blends. These dry blends were produced on a weight basis, depending on the particular application and surgical need. In the present example, the bimodal molecular weight of 85/15 lactide/glycolide copolymer at 75/25 higher Mw/lower Mw in weight percent, were dry blended as described directly below.

Into a clean 3-cubic foot Patterson-Kelley dryer, 2.6 kg of granules of the 85/15 lactide/glycolide copolymer of Example 1 and 0.9 kg of 85/15 lactide/glycolide copolymer of Example 2 were added. The dryer was closed, and the vessel pressure was reduced to less than 200 mTorr. The rotation was started at 7.5 RPM and continued for a minimum period of one hour. The dry blend was then discharged into portable vacuum storage containers, and these containers were placed under vacuum, until ready for the melt blending step.

For the purpose of this invention, blends of this type can be produced in a similar manner with different compositions. Alternately, one may make the inventive blends by combining the Lac/Gly copolymer of normal molecular weight distribution with the Lac/Gly copolymer of lower molecular weight distribution directly in a melt extruder.

### Example 4

### Melt Blending of Unimodal Lactide/Glycolide Copolymers

Once the dry blends were produced and vacuum conditioned for at least three days, the melt-blending step was begun. A ZSK-30 twin-screw extruder was fitted with screws designed for melt blending utilizing dual vacuum ports for purposes of volatilizing residual monomer. The screw design contained several different types of elements, including conveying, compression, mixing and sealing elements. The extruder was fitted with a three-hole die plate, and a chilled water bath with water temperature set between 4.5 and 21° C. was placed near the extruder outlet. A strand pelletizer and pellet classifier was placed at the end of the water bath. The extruder temperature zones were heated to a temperature of 160 to 180° C., and the vacuum cold traps were set to −20° C. The pre-conditioned dry blend granules were removed from vacuum and placed in a twin-screw feed hopper under nitrogen purge. The extruder screws were set to a speed of 175-225 RPM, and the feeder was turned on, allowing the dry blend to be fed into the extruder.

The polymer melt blend was allowed to purge through the extruder until the feed was consistent, at which point the vacuum was applied to the two vacuum ports. The polymer blend extrudate strands were fed through the water bath and into the strand pelletizer. The pelletizer cut the strands into appropriate sized pellets; it was found that pellets with a diameter of 1 mm and an approximate length of 3 mm sufficed. The pellets were then fed into the classifier. The classifier separated substantially oversized and undersized pellets from the desired size, usually a weight of about 10-15 mg per pellet. This process continued until the entire polymer dry blend was melt blended in the extruder, and formed into substantially uniform pellets. Samples were taken throughout the extrusion process and were measured for polymer characteristics such as inherent viscosity, molecular weight and composition. Once the melt-blending process was completed, the pelletized polymer was placed in polyethylene bags, weighed, and stored in a freezer below −20° C. to await devolatilization of residual monomer.

The polymer melt-blend was placed into a 3-cubic foot Patterson-Kelley dryer, which was placed under vacuum. The dryer was closed and the pressure was reduced to less than 200 mTorr. Once the pressure was below 200 mTorr, dryer rotation was activated at a rotational speed of 10 RPM with no heat for 6 hours. After the 6 hour period, the oil temperature was set to 85° C. at a heat up rate of 120° C. per hour. The oil temperature was maintained at 85° C. for a period of 12 hours. At the end of this heating period, the batch was allowed to cool for a period of at least 4 hours, while maintaining rotation and vacuum. The polymer melt-blend pellets were discharged from the dryer by pressurizing the vessel with nitrogen, opening the discharge valve, and allowing the polymer pellets to descend into waiting vessels for long term storage. The storage vessels were air tight and outfitted with valves allowing for evacuation so that the inventive resin blend could be stored under vacuum.

The inventive bimodal molecular weight blend was characterized. The resultant 85/15 lactide/glycolide bimodal melt blend composition exhibited a melt flow index of 0.162 g/10 min, as measured at 190° C. with the standard weight of 6,600 grams. Differential scanning calorimetry of dried pellets revealed a glass transition temperature of 57° C. and a melting transition temperature at 147° C. using a heating rate of 10° C./min. The heat of fusion determined during the first heat (heating rate 10° C./min) was 35.5 J/g.

### Example 5

### Crystallization Kinetics Evaluation of Inventive Bimodal Molecular Weight Blend

Differential Scanning Calorimetry (DSC) was used to investigate the crystallization kinetics of the inventive bimodal molecular weight blend compositions. The following methods/conditions were used:
a) First heat measurements—a 5 to 10 milligram sample of interest was quenched to −60° C. in a DSC pan equipped with nitrogen purge, followed by the constant heating rate scan of 10° C./min.
b) Second heat measurements—the sample of interest after melting in a DSC pan at 185° C., and followed by a rapid quench (−60° C./min) to −60° C. was then heated at the constant heating rate of 5° C./min to 185° C.

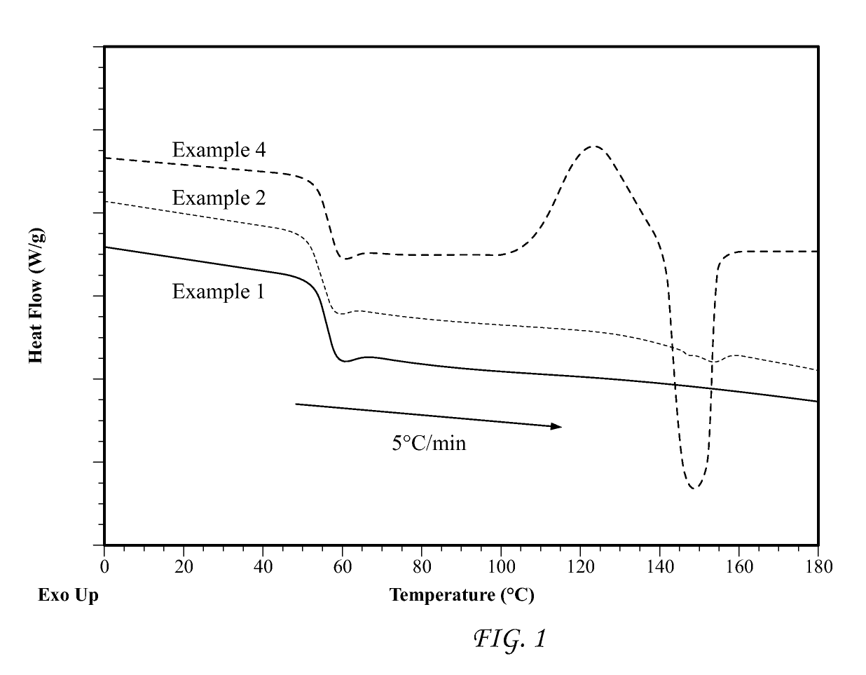

It was unexpectedly discovered that the 85/15 lactide/glycolide bimodal melt blend composition of Example 4 exhibited significantly faster crystallization rate than its individual 85/15 lactide/glycolide components alone. This dramatically faster synergetic effect of the bimodal molecular weight blend is shown in FIG. 1. During the heating step at 5° C./min, the 85/15 lactide/glycolide resin with Mw=90 k (Example 1) exhibited no crystallization, while DSC trace of the lower molecular component (Example 2) showed a very small heat of fusion, AHₘ value (0.7 J/g). In contrast, the bimodal molecular weight blend (Example 4) crystallized rapidly (large peak at about 120° C.), with the heat of fusion value of 15.5 J/g.

A summary of DSC results obtained on pellets of a control and blends of the present invention can be found in Table 1 below. It should be noted that the pellets underwent elevated temperature devolatilization that should have been sufficient to develop a nearly maximum level of crystallinity. This would be reflected in the “first heat” results. The “second heat” results reflect the inherent crystallization properties of the test samples because the thermal history would have been erased, as is well known.

TABLE 1
DSC Calorimetric Properties of Control Samples
and the Inventive Dried Bimodal Blend

| | | First Heat Data | | | Second Heat Data | | |
|---|---|---|---|---|---|---|---|
| | | (10° C./min) | | | (5° C./min) | | |
| | | T_{g} | Tₘ | ΔHₘ | T_{g} | Tₘ | ΔHₘ |
| Sample ID | Comments | (° C.) | (° C.) | (J/g) | (° C.) | (° C.) | (J/g) |
| EXAMPLE | Standard Mw 85/15 | 58.8 | 150 | 35.0 | 55.3 | No | |
| 1 | Lac/Gly (control, non- | | | | | crystallization | |
| | inventive sample) | | | | | | |
| EXAMPLE | Lower Mw 85/15 Lac/Gly | 53.6 | 151 | 41.0 | 53.8 | 153 | 0.7 |
| 2 | (control, non-inventive | | | | | | |
| | sample) | | | | | | |
| EXAMPLE | Bimodal 85/15 Lac/Gly | 57.1 | 147 | 35.5 | 55.3 | 149 | 15.5 |
| 4 | (75/25 high/lower Mw wt. %) | | | | | | |

The advantage of the faster crystallizing bimodal molecular weight blend (Example 4) can be obtained in various melt processing procedures including extrusion, injection molding, blow molding, and similar. Some of the advantages of medical devices made from this inventive resin may include better mechanical properties, higher achievable molecular orientation, less polymer degradation during melt processing, and more economical processes.

### Example 6

### Preparation of 95/5 Poly(L(−)-lactide-co-glycolide) Bimodal Molecular Weight Blend

A standard molecular weight 95/5 poly(L(−)-lactide-co-glycolide) resin was synthesized in the similar fashion as described previously in the Example 1. The NMR results of the dried, annealed copolymer revealed the final chemical composition of about 95 mole % polymerized L(−)-lactide, and about 5 mole % polymerized glycolide. GPC method revealed the weight average molecular weight of about 90,000 Daltons.

Similarly, a lower molecular weight 95/5 poly(L(−)-lactide-co-glycolide) resin was synthesized following the procedures described in Example 2. In this case, however, a higher concentration of initiator was used (100:1 monomer-to-initiator ratio), resulting in the resin having a weight average molecular weight of about 21,000 Daltons, as determined by the GPC method. The final, dried and annealed resin had chemical composition of about 95 mole % L(−)-lactide, and about 5 mole % glycolide as determined by NMR.

Dry and melt blending of unimodal 95/5 poly(L(−)-lactide-co-glycolide) copolymers were conducted following the procedures given in Example 3 and Example 4, respectively. Several different blend compositions were made. The bimodal molecular weight blend used in this example was composed of 80% 95/5 Lac/Gly copolymer with a weight average molecular weight of 90,000 Daltons, and 20% of 95/5 Lac/Gly copolymer having a weight average molecular weight of 21,000 Daltons. Dried and annealed bimodal molecular weight pellets were stored under vacuum until further use.

### Example 7

### Calorimetric Characterization of 95/5 Poly(L(−)-lactide-co-glycolide) Bimodal Molecular Weight Blend (80/20 wt. % Higher/Lower Mw wt. %)

The following DSC methods/conditions were used in characterizing 95/5 Poly(L(−)-lactide-co-glycolide) Bimodal Molecular Weight Blend:
a) First heat measurements—a 5 to 10 milligram sample of interest was quenched to −60° C. in a DSC pan equipped with nitrogen purge, followed by the constant heating rate scan of 10° C./min
b) Second heat measurements—the sample of interest after melting in a DSC pan at 200° C., and followed by a rapid quench (−60° C./min) to −60° C. was then heated at the constant heating rate of 10° C./min to 200° C.

The calorimetric DSC data revealed synergetically faster crystallization kinetics of the 95/5 Poly(L(−)-lactide-co-glycolide) bimodal molecular weight blend compared to corresponding data of its components (standard and lower Mw resin). During the second heat measurement, the values of the heat of crystallization, ΔH_{c} and the heat of fusion, ΔHₘ for the bimodal blend were much higher than for those found on the individual blend components (39 vs. 32/33 J/g). These results are summarized Table 2.

TABLE 2
DSC Calorimetric Properties of 95/5 Lac/Gly resins

| | First Heat (10° C./min) | | | Second Heat (10° C./min) | | | | |
|---|---|---|---|---|---|---|---|---|
| | T_{g} | Tₘ | ΔHₘ | T_{g} | T_{c} | ΔH_{c} | Tₘ | ΔHₘ |
| Polymer ID | (° C.) | (° C.) | (J/g) | (° C.) | (° C.) | (J/g) | (° C.) | (J/g) |
| 95/5 Lac/Gly | 62 | 170 | 39 | 57 | 122 | 32 | 163 | 32 |
| (Unimodal, | | | | | | | | |
| M_{w} = 90k) | | | | | | | | |
| 95/5 Lac/Gly | 55 | 171 | 45 | 53 | 121 | 33 | 166 | 33 |
| (Unimodal, | | | | | | | | |
| M_{w} = 21k) | | | | | | | | |
| 95/5 Lac/Gly | 60 | 165 | 43 | 56 | 116 | 39 | 166 | 39 |
| (80/20 Bimodal | | | | | | | | |
| blend 90k/21k) | | | | | | | | |

The use of fast crystallizing bimodal molecular weight blends is also advantageous during fiber extrusion and drawing processes, such as those used in the manufacture of surgical sutures. Materials exhibiting fast crystallization kinetics generally provide better dimensional stability with greater control of polymer morphology. Drawing of fine fibers is particularly difficult with slow crystallizing polymers, since excessively slow crystallization results in frequently line breaks.

A multifilament (braid) suture of USP size 1 composed of 95/5 poly(L(−)-lactide-co-glycolide) (Lac/Gly) copolymer of standard weight average molecular weight (90,000 Daltons) was produced, and a braid of the same size using the inventive bimodal molecular weight blend composed of 80 wt % 95/5 Lac/Gly copolymer with a weight average molecular weight of 90,000 Daltons, and 20 wt % of 95/5 Lac/Gly copolymer having a weight average molecular weight of 21,000 Daltons (Example 6). The extrusion and braiding procedures used to make these fibers were described in U.S. Pat. Nos. 6,756,000 and U.S. Pat. No. 6,743,505 respectively, incorporated herein by reference in their entireties. The multifilament extrusion of the bimodal blend proceeded smoothly, resulting in a tensile strength of the annealed fiber only about 5% lower than the corresponding 95/5 Lac/Gly unimodal braid (Mw=90 k) of the same size. As was the case with the dried resins, calorimetric data revealed faster crystallization kinetics, and also higher crystallinity level in the bimodal molecular weight braid compared to that of the control (Unimodal, Mw=90,000 Daltons).

### Example 8

### Hydrolysis Profile of the Fiber Braid Made from 95/5 Poly(L(−)-lactide-co-glycolide) Bimodal Molecular Weight Blend

The hydrolysis profile properties of the USP size 1 fiber braids made from the inventive 95/5 poly(L(−)-lactide-co-glycolide) (Lac/Gly) bimodal molecular weight blend and the 95/5 Lac/Gly copolymer having unimodal, standard molecular weight distribution (90,000 Daltons) were evaluated.

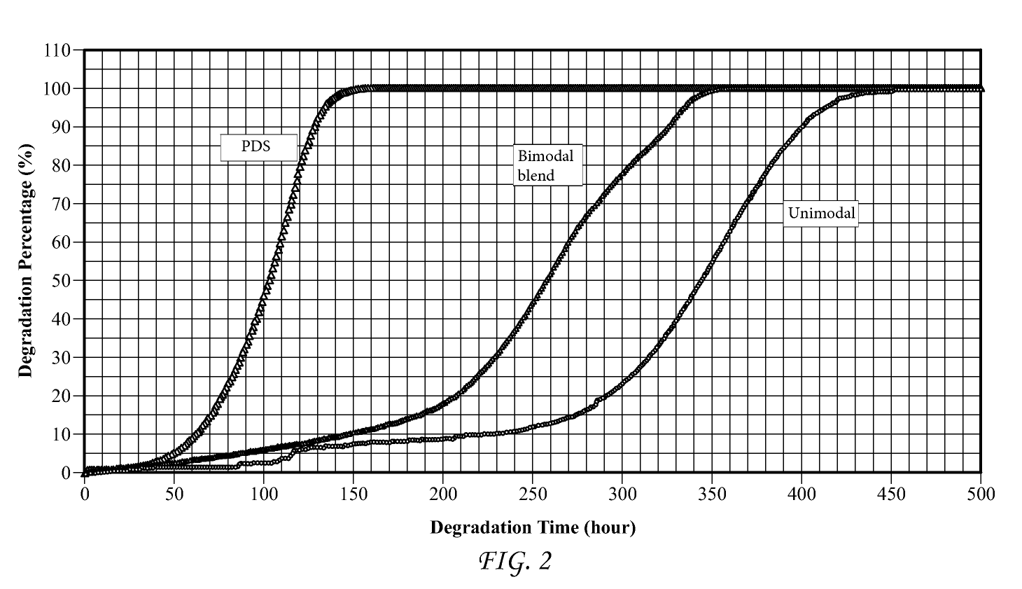

The dried and annealed braids were subjected to in vitro hydrolytic degradation in a buffer at 70° C. with pH maintained at 7.3. The hydrolysis data are presented in FIG. 2. As an additional comparison, the hydrolysis results of a PDS II monofilament (made from Poly(p-dioxanone) homopolymer) of normal molecular weight distribution (FIG. 2). Surprisingly, it was found that a braid made from the faster crystallizing 95/5 Lac/Gly bimodal molecular weight blend (80/20 wt. %, 90 k/21 k) also hydrolyzes considerably faster than the fiber made from 95/5 Lac/Gly unimodal molecular weight distribution. For instance, the half-time of hydrolysis (the time needed to achieve 50% of total hydrolysis) for PDS II monofilament at these in vitro conditions was found to be around 100 hours, for unimodal 95/5 Lac/Gly braid this parameter was around 350 hours, but for bimodal 95/5 Lac/Gly braid that time was reduced significantly to only about 260 hours.

The significance of this finding is that the use of bimodal molecular weight blend approach simultaneously provides opportunity to make a medical device (e.g., sutures) which will have both improved mechanical properties and a shorter total absorption time. This is of particular importance for medical devices used in surgical procedures where wound healing is fast and where prolonged existence of a device may cause patient discomfort. Procedures that demand the absolute best aesthetic outcome may also benefit from the faster hydrolysis profile, as long-lasting medical devices may, on occasion, induce unwanted foreign body reactions.

PCT1. A bimodal polymer composition, comprising (a) a first amount of a first poly(L-lactide-co-glycolide) copolymer having a first crystallization rate, a first hydrolysis rate and a first molecular weight distribution; and (b) a second amount of a second poly(L-lactide-co-glycolide) copolymer having a second crystallization rate, a second hydrolysis rate and a second molecular weight distribution and a weight average molecular weight from about 10,000 to about 50,000 Daltons; wherein the weight average molecular weight ratio of said first molecular weight distribution to said second molecular weight distribution is at least about two to one; and wherein a substantially homogeneous blend of said first and second copolymers is formed in a ratio of between about 50/50 to about 95/5 weight/weight percent, said substantially homogeneous blend having a crystallization rate greater than each of said first crystallization rate and said second crystallization rate and a hydrolysis rate greater than each of said first hydrolysis rate and said second hydrolysis rate.

PCT2. The bimodal polymer composition of paragraph PCT1, having a heat of fusion value of about 15 to about 50 J/g after melt-processing or heat treating the composition, as measured by differential scanning calorimetry using the heating rate of 10° C./min.

PCT3. The bimodal polymer composition of paragraph PCT1 or PCT2, wherein the first and second copolymers comprise from about 80 mol % to about 99 mol % L-lactide and about 1 mol % to about 20 mol % glycolide.

PCT4. The bimodal polymer composition of any one of paragraphs PCT1 to PCT3, wherein the first and second copolymers comprise about 85 mol % L-lactide and about 15 mol % glycolide, or about 95 mol % L-lactide and about 5 mol % glycolide.

PCT5. The bimodal polymer composition of any one of paragraphs PCT1 to PCT4, wherein said first molecular weight distribution is a weight average molecular weight from about 50,000 to about 2,000,000 Daltons.

PCT6. The bimodal polymer composition of any one of paragraphs PCT1 to PCT5, wherein said first amount is from about 70 wt % to about 80 wt % and the second amount is from about 20 wt % to about 30 wt %.

PCT7. The bimodal polymer composition of any one of paragraphs PCT1 to PCT6, wherein said first copolymer has no measurable crystallinity during the second heating scan, as measured by differential scanning calorimetry at a heating rate of 5° C./min.

PCT8. A bimodal polymer composition, comprising (a) from about 70 wt % to about 80 wt % of a first poly(L-lactide-co-glycolide) copolymer having a first crystallization rate, a first hydrolysis rate and a weight average molecular weight from about 50,000 to about 2,000,000 Daltons; and (b) from about 20 wt % to about 30 wt % of a second poly(L-lactide-co-glycolide) copolymer having a second crystallization rate, a second hydrolysis rate and a second molecular weight distribution and a weight average molecular weight between about 10,000 to about 50,000 Daltons; wherein the weight average molecular weight ratio of said first copolymer to said second copolymer is at least about two to one; and wherein a substantially homogeneous blend of said first and second copolymers has a crystallization rate greater than each of said first crystallization rate and said second crystallization rate and a hydrolysis rate greater than each of said first hydrolysis rate and said second hydrolysis rate.

PCT9. A medical device comprising a bimodal polymer composition of (a) a first amount of a first poly(L-lactide-co-glycolide) copolymer having a first crystallization rate, a first hydrolysis rate and a first molecular weight distribution; and (b) a second amount of a second poly(L-lactide-co-glycolide) copolymer having a second crystallization rate, a second hydrolysis rate and a second molecular weight distribution and a weight average molecular weight from about 10,000 to about 50,000 Daltons; wherein the weight average molecular weight ratio of said first molecular weight distribution to said second molecular weight distribution is at least about two to one; and wherein a substantially homogeneous blend of said first and second copolymers is formed in a ratio of between about 50/50 to about 95/5 weight/weight percent, said substantially homogeneous blend having a crystallization rate greater than each of said first crystallization rate and said second crystallization rate and a hydrolysis rate greater than each of said first hydrolysis rate and said second hydrolysis rate

PCT10. The medical device of paragraph PCT9, wherein the first and second copolymers comprise about 85 mol % L-lactide and about 15 mol % glycolide, said first amount is from about 70 wt % to about 80 wt % and the second amount is from about 20 wt % to about 30 wt %.

PCT11. The medical device of paragraph PCT9 or PCT10, the bimodal polymer composition thereof having a heat of fusion value of about 15 to about 50 J/g after melt-processing or heat treating the device over a temperature range of between about 85° C. to about 150° C., as measured by differential scanning calorimetry using the heating rate of 10° C./min.

PCT12. The medical device of any one of paragraphs PCT9 to PCT11, which is a suture, a clip, a staple, a pin, a screw, a fiber, a fabric, a mesh, a clamp, a plate, a hook, a button, a snap, a prosthetic, a graft, an injectable polymer, a vertebrae disc, an anchoring device, a suture anchor, a septal occlusion device, an injectable defect filler, a preformed defect filler, a bone wax, a cartilage replacement, a spinal fixation device, a drug delivery device, a foam or a film.

PCT13. A method of making a bimodal, semi-crystalline poly(L-lactide-co-glycolide) copolymer blend, comprising blending between about 50/50 to about 95/5 weight/weight percent of (1) a first amount of a first poly(L-lactide-co-glycolide) copolymer having a first crystallization rate, a first hydrolysis rate and a first molecular weight distribution, with (2) a second amount of a second poly(L-lactide-co-glycolide) copolymer having a second crystallization rate, a second hydrolysis rate and a second molecular weight distribution and a weight average molecular weight from about 10,000 to about 50,000 Daltons, wherein the weight average molecular weight ratio of said first molecular weight distribution to said second molecular weight distribution is at least about two to one, said blend has a crystallization rate greater than each of said first crystallization rate and said second crystallization rate and a hydrolysis rate greater than each of said first hydrolysis rate and said second hydrolysis rate, and melt-processing or heat treating the blended copolymers over a temperature range of between about 85° C. to about 150° C.

PCT14. The method of making a bimodal, semi-crystalline poly(L-lactide-co-glycolide) copolymer blend of paragraph PCT13, wherein the resulting semi-crystalline poly(L-lactide-co-glycolide) copolymer blend has a heat of fusion value of about 15 to about 50 J/g after melt-processing or heat treating the composition, as measured by differential scanning calorimetry using the heating rate of 10° C./min.

PCT15. The method of making a bimodal, semi-crystalline poly(L-lactide-co-glycolide) copolymer blend of paragraph PCT13 or PCT14, wherein the first and second copolymers comprise from about 85 mol % to about 95 mol % L-lactide and from about 5 mol % to about 15 mol % glycolide, said first amount is from about 70 wt % to about 80 wt % and the second amount is from about 20 wt % to about 30 wt %.

PCT16. The method of making a bimodal, semi-crystalline poly(L-lactide-co-glycolide) copolymer blend of any one of paragraphs PCT13 to PCT15, wherein melt-processing includes melt blending, extruding, melt spinning, melt blowing or injection molding the blended first and second copolymers at a temperature above their melting temperatures, followed by cooling and crystallizing the blend.

PCT17. A method of making a medical device, comprising blending between about 50/50 to about 95/5 weight/weight percent of (1) a first amount of a first poly(L-lactide-co-glycolide) copolymer having a first crystallization rate, a first hydrolysis rate and a first molecular weight distribution, with (2) a second amount of a second poly(L-lactide-co-glycolide) copolymer having a second crystallization rate, a second hydrolysis rate and a second molecular weight distribution and a weight average molecular weight from about 10,000 to about 50,000 Daltons, to form a bimodal, blended copolymer, wherein the weight average molecular weight ratio of said first molecular weight distribution to said second molecular weight distribution is at least about two to one, said blend has a crystallization rate greater than each of said first crystallization rate and said second crystallization rate and a hydrolysis rate greater than each of said first hydrolysis rate and said second hydrolysis rate, and forming the medical device by melt-processing or heat treating the blended copolymer over a temperature range of between about 85° C. to about 150° C.

PCT18. The method of making a medical device of paragraph PCT17, wherein the bimodal, blended copolymer of the medical device has a heat of fusion value of about 15 to about 50 J/g after melt-processing or heat treating the composition, as measured by differential scanning calorimetry using the heating rate of 10° C./min.

PCT19. The method of making a medical device of paragraph PCT17 or PCT18, wherein the first and second copolymers comprise from about 85 mol % to about 95 mol % L-lactide and from about 5 mol % to about 15 mol % glycolide, said first amount is from about 70 wt % to about 80 wt % and the second amount is from about 20 wt % to about 30 wt %.

PCT20. The method of making a medical device of any one of paragraphs PCT17 to PCT19, wherein melt-processing includes melt blending, extruding, melt spinning, melt blowing or injection molding the blended first and second copolymers at a temperature above their melting temperatures, followed by cooling and crystallizing the blend.

PCT21. The method of making a medical device of any one of paragraphs PCT17 to PCT20, wherein the medical device is a suture, a clip, a staple, a pin, a screw, a fiber, a fabric, a mesh, a clamp, a plate, a hook, a button, a snap, a prosthetic, a graft, an injectable polymer, a vertebrae disc, an anchoring device, a suture anchor, a septal occlusion device, an injectable defect filler, a preformed defect filler, a bone wax, a cartilage replacement, a spinal fixation device, a drug delivery device, a foam or a film.

PCT22. A semi-crystalline polymer composition, comprising a blend of from about 50 to about 95 wt % of a first poly(L-lactide-co-glycolide) copolymer having a first weight average molecular weight distribution; and from about 50 to about 5 wt % of a second poly(L-lactide-co-glycolide) copolymer having a second weight average molecular weight distribution from about 10,000 to about 50,000 Daltons; wherein the ratio of said first molecular weight distribution to said second molecular weight distribution is at least about two to one, and said blend has a crystallization rate greater than crystallization rates of both said first and second copolymers.

While the subject invention has been illustrated and described in detail in the drawings and foregoing description, the disclosed embodiments are illustrative and not restrictive in character. All changes and modifications that come within the scope of the invention are desired to be protected.

1. A bimodal polymer composition, comprising:
(a) a first amount of a first poly(L-lactide-co-glycolide) copolymer having a first crystallization rate, a first hydrolysis rate and a first molecular weight distribution; and
(b) a second amount of a second poly(L-lactide-co-glycolide) copolymer having a second crystallization rate, a second hydrolysis rate and a second molecular weight distribution and a weight average molecular weight from about 10,000 to about 50,000 Daltons;
wherein the weight average molecular weight ratio of said first molecular weight distribution to said second molecular weight distribution is at least about two to one; and
wherein a substantially homogeneous blend of said first and second copolymers is formed in a ratio of between about 50/50 to about 95/5 weight/weight percent, said substantially homogeneous blend having a crystallization rate greater than each of said first crystallization rate and said second crystallization rate and a hydrolysis rate greater than each of said first hydrolysis rate and said second hydrolysis rate.

2. The bimodal polymer composition of claim 1, having a heat of fusion value of about 15 to about 50 J/g after melt-processing or heat treating the composition, as measured by differential scanning calorimetry using the heating rate of 10° C./min.

3. The bimodal polymer composition of claim 1, wherein the first and second copolymers comprise from about 80 mol % to about 99 mol % L-lactide and about 1 mol % to about 20 mol % glycolide.

4. The bimodal polymer composition of claim 1, wherein the first and second copolymers comprise about 85 mol % L-lactide and about 15 mol % glycolide.

5. The bimodal polymer composition of claim 1, wherein the first and second copolymers comprise about 95 mol % L-lactide and about 5 mol % glycolide.

6. The bimodal polymer composition of claim 1, wherein said first molecular weight distribution is a weight average molecular weight from about 50,000 to about 2,000,000 Daltons.

7. The bimodal polymer composition of claim 1, wherein said first amount is from about 70 wt % to about 80 wt % and the second amount is from about 20 wt % to about 30 wt %.

8. The bimodal polymer composition of claim 1, wherein said first copolymer has no measurable crystallinity during the second heating scan, as measured by differential scanning calorimetry at a heating rate of 5° C./min.

9. A bimodal polymer composition, comprising:
(a) from about 70 wt % to about 80 wt % of a first poly(L-lactide-co-glycolide) copolymer having a first crystallization rate, a first hydrolysis rate and a weight average molecular weight from about 50,000 to about 2,000,000 Daltons; and
(b) from about 20 wt % to about 30 wt % of a second poly(L-lactide-co-glycolide) copolymer having a second crystallization rate, a second hydrolysis rate and a second molecular weight distribution and a weight average molecular weight between about 10,000 to about 50,000 Daltons;
wherein the weight average molecular weight ratio of said first copolymer to said second copolymer is at least about two to one; and
wherein a substantially homogeneous blend of said first and second copolymers has a crystallization rate greater than each of said first crystallization rate and said second crystallization rate and a hydrolysis rate greater than each of said first hydrolysis rate and said second hydrolysis rate.

10. A medical device comprising a bimodal polymer composition of:
(a) a first amount of a first poly(L-lactide-co-glycolide) copolymer having a first crystallization rate, a first hydrolysis rate and a first molecular weight distribution; and
(b) a second amount of a second poly(L-lactide-co-glycolide) copolymer having a second crystallization rate, a second hydrolysis rate and a second molecular weight distribution and a weight average molecular weight from about 10,000 to about 50,000 Daltons;
wherein the weight average molecular weight ratio of said first molecular weight distribution to said second molecular weight distribution is at least about two to one; and
wherein a substantially homogeneous blend of said first and second copolymers is formed in a ratio of between about 50/50 to about 95/5 weight/weight percent, said substantially homogeneous blend having a crystallization rate greater than each of said first crystallization rate and said second crystallization rate and a hydrolysis rate greater than each of said first hydrolysis rate and said second hydrolysis rate.

11. The medical device of claim 10, wherein the first and second copolymers comprise about 85 mol % L-lactide and about 15 mol % glycolide, said first amount is from about 70 wt % to about 80 wt % and the second amount is from about 20 wt % to about 30 wt %.

12. The medical device of claim 10, the bimodal polymer composition thereof having a heat of fusion value of about 15 to about 50 J/g after melt-processing or heat treating the device over a temperature range of between about 85° C. to about 150° C., as measured by differential scanning calorimetry using the heating rate of 10° C./min.

13. The medical device of claim 10, which is a suture, a clip, a staple, a pin, a screw, a fiber, a fabric, a mesh, a clamp, a plate, a hook, a button, a snap, a prosthetic, a graft, an injectable polymer, a vertebrae disc, an anchoring device, a suture anchor, a septal occlusion device, an injectable defect filler, a preformed defect filler, a bone wax, a cartilage replacement, a spinal fixation device, a drug delivery device, a foam or a film.

14. A method of making a bimodal, semi-crystalline poly(L-lactide-co-glycolide) copolymer blend, comprising:
blending between about 50/50 to about 95/5 weight/weight percent of:
(1) a first amount of a first poly(L-lactide-co-glycolide) copolymer having a first crystallization rate, a first hydrolysis rate and a first molecular weight distribution, with
(2) a second amount of a second poly(L-lactide-co-glycolide) copolymer having a second crystallization rate, a second hydrolysis rate and a second molecular weight distribution and a weight average molecular weight from about 10,000 to about 50,000 Daltons,
wherein the weight average molecular weight ratio of said first molecular weight distribution to said second molecular weight distribution is at least about two to one, said blend has a crystallization rate greater than each of said first crystallization rate and said second crystallization rate and a hydrolysis rate greater than each of said first hydrolysis rate and said second hydrolysis rate, and
melt-processing or heat treating the blended copolymers over a temperature range of between about 85° C. to about 150° C.

15. The method of claim 14, wherein the resulting semi-crystalline poly(L-lactide-co-glycolide) copolymer blend has a heat of fusion value of about 15 to about 50 J/g after melt-processing or heat treating the composition, as measured by differential scanning calorimetry using the heating rate of 10° C./min.

16. The method of claim 14, wherein the first and second copolymers comprise from about 85 mol % to about 95 mol % L-lactide and from about 5 mol % to about 15 mol % glycolide, said first amount is from about 70 wt % to about 80 wt % and the second amount is from about 20 wt % to about 30 wt %.

17. The method of claim 14, wherein melt-processing includes melt blending, extruding, melt spinning, melt blowing or injection molding the blended first and second copolymers at a temperature above their melting temperatures, followed by cooling and crystallizing the blend.

18. A method of making a medical device, comprising:
blending between about 50/50 to about 95/5 weight/weight percent of:
(1) a first amount of a first poly(L-lactide-co-glycolide) copolymer having a first crystallization rate, a first hydrolysis rate and a first molecular weight distribution, with
(2) a second amount of a second poly(L-lactide-co-glycolide) copolymer having a second crystallization rate, a second hydrolysis rate and a second molecular weight distribution and a weight average molecular weight from about 10,000 to about 50,000 Daltons, to form a bimodal, blended copolymer,
wherein the weight average molecular weight ratio of said first molecular weight distribution to said second molecular weight distribution is at least about two to one, said blend has a crystallization rate greater than each of said first crystallization rate and said second crystallization rate and a hydrolysis rate greater than each of said first hydrolysis rate and said second hydrolysis rate, and
forming the medical device by melt-processing or heat treating the blended copolymer over a temperature range of between about 85° C. to about 150° C.

19. The method of claim 18, wherein the bimodal, blended copolymer of the medical device has a heat of fusion value of about 15 to about 50 J/g after melt-processing or heat treating the composition, as measured by differential scanning calorimetry using the heating rate of 10° C./min.

20. The method of claim 18, wherein the first and second copolymers comprise from about 85 mol % to about 95 mol % L-lactide and from about 5 mol % to about 15 mol % glycolide, said first amount is from about 70 wt % to about 80 wt % and the second amount is from about 20 wt % to about 30 wt %.

21. The method of claim 18, wherein melt-processing includes melt blending, extruding, melt spinning, melt blowing or injection molding the blended first and second copolymers at a temperature above their melting temperatures, followed by cooling and crystallizing the blend.

22. The method of claim 18, wherein the medical device is a suture, a clip, a staple, a pin, a screw, a fiber, a fabric, a mesh, a clamp, a plate, a hook, a button, a snap, a prosthetic, a graft, an injectable polymer, a vertebrae disc, an anchoring device, a suture anchor, a septal occlusion device, an injectable defect filler, a preformed defect filler, a bone wax, a cartilage replacement, a spinal fixation device, a drug delivery device, a foam or a film.

23. A semi-crystalline polymer composition, comprising a blend of:
from about 50 to about 95 wt % of a first poly(L-lactide-co-glycolide) copolymer having a first weight average molecular weight distribution; and
from about 50 to about 5 wt % of a second poly(L-lactide-co-glycolide) copolymer having a second weight average molecular weight distribution from about 10,000 to about 50,000 Daltons;
wherein the ratio of said first molecular weight distribution to said second molecular weight distribution is at least about two to one, and said blend has a crystallization rate greater than crystallization rates of both said first and second copolymers.
